SEQUENCE LISTING (14 sequences)

SEQ ID NO: 1 - DNA, 5839 bp, Vesicularia sp.
  source 1..5839: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccgccaccatggaaacagataccttgcttctctgggtgctgctcttgtgggtccccggatcaacaggggaacagaagctgatcagcgaggaggatctgcaggttcagttggtagaaagcggtggcaatttggtccagcctggcggatctttgcgcctttcctgtgctgcatcaggctttacttttgggtctttctcaatgtcctgggtaaggcaagccccaggaggcggactcgagtgggtagccggactgtcagctcgatcctccctcactcattatgccgactccgttaagggacggtttaccatctccagggataatgccaaaaatagcgtctacctccaaatgaactctttgcgagtggaagatacagccgtctactattgcgcccgcaggagctacgattcaagcggctattggggtcatttctattcctacatggacgtttggggccaagggaccctcgtgaccgttagctcaggagggggtggtagcgggggaggagggagtgggggcggtggaagccagtctgtgctcacccaaccatcttcagtaagtgcagcaccagggcaaaaggtaactatttcctgtagtgggtctacttccaatataggtaacaactacgtgtcatggtaccagcaacacccaggcaaagcacccaaattgatgatctacgacgtgtcaaaacgcccctcaggggtacctgatcgattttcaggttctaaatctgggaatagtgcttcacttgatatctcaggtctgcaaagcgaagatgaagcagattactactgcgctgcatgggacgattccttgtctgaatttcttttcggaactggcaccaaactgaccgtattggggaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 2 - DNA, 1044 bp, synthetic construct
  source 1..1044: mol_type = other DNA
atggaaacagataccttgcttctctgggtgctgctcttgtgggtccccggatcaacaggggaacagaagctgatcagcgaggaggatctgcaggttcagttggtagaaagcggtggcaatttggtccagcctggcggatctttgcgcctttcctgtgctgcatcaggctttacttttgggtctttctcaatgtcctgggtaaggcaagccccaggaggcggactcgagtgggtagccggactgtcagctcgatcctccctcactcattatgccgactccgttaagggacggtttaccatctccagggataatgccaaaaatagcgtctacctccaaatgaactctttgcgagtggaagatacagccgtctactattgcgcccgcaggagctacgattcaagcggctattggggtcatttctattcctacatggacgtttggggccaagggaccctcgtgaccgttagctcaggagggggtggtagcgggggaggagggagtgggggcggtggaagccagtctgtgctcacccaaccatcttcagtaagtgcagcaccagggcaaaaggtaactatttcctgtagtgggtctacttccaatataggtaacaactacgtgtcatggtaccagcaacacccaggcaaagcacccaaattgatgatctacgacgtgtcaaaacgcccctcaggggtacctgatcgattttcaggttctaaatctgggaatagtgcttcacttgatatctcaggtctgcaaagcgaagatgaagcagattactactgcgctgcatgggacgattccttgtctgaatttcttttcggaactggcaccaaactgaccgtattggggaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 3 - DNA, 60 bp, synthetic construct
  source 1..60: mol_type = other DNA
atggaaacagataccttgcttctctgggtgctgctcttgtgggtccccggatcaacaggg

SEQ ID NO: 4 - DNA, 25 bp, synthetic construct
  source 1..25: mol_type = other DNA
gaagctgatcagcgaggaggatctg

SEQ ID NO: 5 - DNA, 759 bp, synthetic construct
  source 1..759: mol_type = other DNA
caggttcagttggtagaaagcggtggcaatttggtccagcctggcggatctttgcgcctttcctgtgctgcatcaggctttacttttgggtctttctcaatgtcctgggtaaggcaagccccaggaggcggactcgagtgggtagccggactgtcagctcgatcctccctcactcattatgccgactccgttaagggacggtttaccatctccagggataatgccaaaaatagcgtctacctccaaatgaactctttgcgagtggaagatacagccgtctactattgcgcccgcaggagctacgattcaagcggctattggggtcatttctattcctacatggacgtttggggccaagggaccctcgtgaccgttagctcaggagggggtggtagcgggggaggagggagtgggggcggtggaagccagtctgtgctcacccaaccatcttcagtaagtgcagcaccagggcaaaaggtaactatttcctgtagtgggtctacttccaatataggtaacaactacgtgtcatggtaccagcaacacccaggcaaagcacccaaattgatgatctacgacgtgtcaaaacgcccctcaggggtacctgatcgattttcaggttctaaatctgggaatagtgcttcacttgatatctcaggtctgcaaagcgaagatgaagcagattactactgcgctgcatgggacgattccttgtctgaatttcttttcggaactggcaccaaactgaccgtattgggg

SEQ ID NO: 6 - DNA, 60 bp, synthetic construct
  source 1..60: mol_type = other DNA
aaaaatccaatcgagcttgtagaaggttggttcagtagttggaaaagctctattgcctct

SEQ ID NO: 7 - DNA, 45 bp, synthetic construct
  source 1..45: mol_type = other DNA
tttttctttatcatagggttaatcattggactattcttggttctc

SEQ ID NO: 8 - protein, 347 aa, synthetic construct
METDTLLLWVLLLWVPGSTGEQKLISEEDLQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSGGGGSGGGGSGGGGSQSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGKNPIELVEGWFSSWKSSIASFFFIIGLIIGLFLVLRVGIHLCIKLKHTKKRQIYTDIEMNRLGK

SEQ ID NO: 9 - protein, 20 aa, synthetic construct
METDTLLLWVLLLWVPGSTG

SEQ ID NO: 10 - protein, 10 aa, synthetic construct
EQKLISEEDL

SEQ ID NO: 11 - protein, 253 aa, synthetic construct
QVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSGGGGSGGGGSGGGGSQSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLG

SEQ ID NO: 12 - protein, 20 aa, synthetic construct
KNPIELVEGWFSSWKSSIAS

SEQ ID NO: 13 - protein, 15 aa, synthetic construct
FFFIIGLIIGLFLVL

SEQ ID NO: 14 - protein, 29 aa, synthetic construct
RVGIHLCIKLKHTKKRQIYTDIEMNRLGK

(19) United States
(12) Patent Application Publication
(10) Pub. No.: US 2026/0308617 A1
(43) Pub. Date: Oct. 8, 2026

(54) CELL SURFACE ENGINEERED OF TAILORED THERAPEUTIC GENE DELIVERY

(71) Applicants: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(72) Inventors: Leyuan MA, Philadelphia, PA (US); Letitia CHAN, Philadelphia, PA (US)

(73) Assignees: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(21) Appl. No.: 19/479,859

(22) PCT Filed: Jun. 28, 2024
(86) PCT No.: PCT/US2024/035972

§ 371 Date: Oct. 29, 2025

Related U.S. Application Data
(60) Provisional application No. 63/511,468, filed on Jun. 30, 2023.

(51) Int. Cl. C12N 15/86; C07K 14/005; C07K 16/44

(52) U.S. Cl. CPC C12N 15/86; C07K 14/005; C07K 16/44; C07K 2317/622; C07K 2319/00; C12N 2740/16043; C12N 2740/16045; C12N 2760/20222

(57) ABSTRACT

The present disclosure is directed to methods of improving the transduction efficiency of non-transducible cells, such as cells with low hLDLR. The cells may be labeled with 5FAM and transduced with a scFv-truncated VSV-G pseudotyped lentivirus which comprises an anti-fluorescein scFv. Further provided are methods of gene therapy, cell engineering, genetic screening, and disease models using the fluorescein-labeled cells transduced with the scFv-truncated VSV-G pseudotyped lentivirus.

### PRIORITY CLAIM

This application claims benefit of priority to U.S. Provisional Application Ser. No. 63/511,468, filed Jun. 30, 2023, the entire contents of which are hereby incorporated by reference.

### SEQUENCE LISTING DISCLOSURE

This application contains a Sequence Listing XML, which has been submitted electronically and is hereby incorporated by reference in its entirety. Said XML Sequence Listing, created on Jun. 14, 2024, is named CHOPP0069WO.xml and is 21,586 bytes in size.

### BACKGROUND

### 1. Field of the Disclosure

The present disclosure relates generally to the fields of molecular biology. More particularly, the disclosure relates to improved methods of transduction, such as for therapeutic gene delivery.

### 2. Background

In current laboratory practice, transduction efficiency of certain cell types using VSV-G pseudotyped lentiviruses is quite low. For example, in murine T cells the transduction efficiency ranges from 5-10%. In canine hematopoietic cells, there is also low transduction efficiency, typically less than 1%. Given that many CRISPR libraries and genetic engineering tools are all lentivirus-based, there is an unmet need to expand the application of these tools to a wider variety of cell types and animal models for immunology research as well as pre-clinical evaluation of cell-based immunotherapies.

### SUMMARY

Thus, in accordance with the present disclosure, there is a composition:
comprising a cell conjugated to fluorescein, wherein the cell has low or essentially no expression or undetectable expression of human low density lipoprotein receptor (hLDLR), such as wherein the cell has low, undetectable or essentially no expression of human low density lipoprotein receptor (hLDLR), such as measured by flow cytometry; or
comprising a cell expressing a VSV-G chimeric protein conjugated to conjugated to fluorescein.

The fluorescein may be 5-carboxyfluorescein (5-FAM), FITC, or 6-carboxyfluorescein (6′FAM). The fluorescein may be NHS-5′FAM, Maleimide-5′FAM, or 1,2-Distearoyl-sn-glycero-3-phosphoethanolamine (DSPE)-Poly-ethylene-glycol(PEG)-Fluorescein. The cell with low, undetectable or essentially no expression of hLDLR may a human naïve T cell, resting human T cells, a human B cell, human epithelial cell, murine immune cell, or canine immune cell, monkey immune cell, or cancer cell line (e.g., SUP-B15 or Raji). The murine immune cell, monkey immune cell, or canine immune cell may be a T cell, NK cell, or a hematopoietic cell. The 5-FAM may conjugated to N-hydroxysuccinimide at the 5′ carbon (NHS-5′-FAM) or may be is conjugated to an antibody targeting a cell surface protein, such as wherein the cell surface protein is CD71 on K562 cells, CD3, CD4, CD8, CD5, or CD7 on T cells, or CD19, CD20, or CD22 on B cells. The cell may be have been transduced with a pseudotyped lentivirus, such as a scFv-truncated vesicular stomatitis virus envelope glycoprotein (VSV-G) pseudotyped lentivirus, e.g., wherein the VSV-G pseudotyped lentivirus comprises a VSV-G hinge, intracellular domain and transmembrane domain fused to an anti-fluorescein scFv. The anti-fluorescein scFv may be positioned at the N terminal of the lentivirus. The anti-fluorescein scFv may comprise 2A9M derived from fluorescein targeting antibody FITC-E2.

A further embodiment provides a nucleic acid encoding a VSV-G chimeric protein linked to an anti-fluorescein antibody or fragment thereof. In some aspects, the anti-fluorescein antibody is an anti-fluorescein scFv. In some aspects, the anti-fluorescein scFv comprises the antibody clone FITC-E2, 4m5.3, or 4-4-20.

In certain aspects, the anti-fluorescein scFv is positioned at the N terminus of the chimeric protein. In some aspects, the anti-fluorescein scFv is a 2A9M scFv derived from fluorescein targeting antibody FITC-E2. In certain aspects, the anti-fluorescein is fused to a VSV-G hinge, transmembrane and intracellular domain. In some aspects, the fusion protein is under control of a constitutive promoter. In certain aspects, the constitutive promoter is CMV, CAG, or EF1a.

Another embodiment provides a vector comprising the nucleic acid of the present embodiments or aspects thereof (e.g., a cell conjugated to 5-carboxyfluorescein (5-FAM), wherein the cell has low or essentially no expression of human low density lipoprotein receptor (hLDLR)). In some aspects, the vector is a HIV-1-derived lentiviral vector.

Also provided herein is a host cell comprising the nucleic acid of the present embodiments or aspects thereof (e.g., a nucleic acid encoding a VSV-G chimeric protein linked to an anti-fluorescein antibody or fragment thereof). A further embodiment provides a polypeptide encoded by the nucleic acid of any one of the present embodiments or aspects thereof (e.g., a nucleic acid encoding a VSV-G chimeric protein linked to an anti-fluorescein antibody or fragment thereof).

Another embodiment provides a pseudotyped lentiviral vector particle comprising a VSV-G envelope and the polypeptide comprising a VSV-G chimeric protein linked to an anti-fluorescein antibody or fragment thereof.

A further embodiment provides a method for transducing a cell comprising contacting said cell with the pseudotyped lentiviral vector particles of the present embodiments (e.g., a pseudotyped lentiviral vector particle comprising a VSV-G envelope and the polypeptide comprising a VSV-G chimeric protein linked to an anti-fluorescein antibody or fragment thereof under conditions suitable for transduction, thereby transducing said cell.

In some aspects, the cell is a cell according to the present embodiments or aspects thereof (e.g., a host cell comprising a nucleic acid encoding a VSV-G chimeric protein linked to an anti-fluorescein antibody or fragment thereof). In some aspects, the contacting is in the present of a cationic polymer. In certain aspects, the cationic polymer is polybrene.

In some aspects, the method further comprises concentrating viral supernatant by high-speed centrifugation. In certain aspects, the high-speed centrifugation is performed at more than 15,000 rpm. In certain aspects, centrifugation is performed for 15 to 30 hours. In specific aspects, the centrifugation is performed for 20 hours. In some aspects, the transduction efficiency is at least 30%, 40%, 50%, 60%, 70%, 80%, or 90%.

Further provided herein are methods for use of the chemically labeled cells transduced with pseudotyped lentiviruses for gene therapy and lentivirus-based in vivo gene delivery, human naïve T cell engineering, and genetic screens using murine T cells for T cell immunology and cancer immunology research.

In additional embodiments, the present lentivirus can be used for pre-clinical studies using the customized lentiviruses or a lentivirus library for genomic screens (e.g., RNAi, CRISPR, ORF library). For example, the desired non-transducible cells can be surface-labeled with fluorescein followed by the addition of VSVG/aFITC dual-envelope pseudotyped lentivirus using routine transduction method (e.g., spin transduction). Further embodiments provide methods for using the present lentivirus for pre-clinical studies using a gene-modified mouse, canine, or monkey T cells. Another embodiment provides use of naïve, resting or activated human T cells pre-labeled with fluorescein followed by addition of VSVG/aFITC dual envelope pseudotyped lentivirus using routine transduction method (e.g., spin transduction) for gene therapy.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: Schematics comparing lentiviral transduction of the standard low hLDLR expressing cells with transduction of chemically labelled cells by scFv pseudotyped lentiviruses.

FIG. 2: Schematic showing genetic engineering of scFv-truncVSVG, used in pseudotyping of lentiviral particles. The N terminus consists of the 2A9M scFv, derived from fluoresecein targeting antibody FITCE2, followed by the hinge, transmembrane, and intracellular domains of VSVG respectively.

FIGS. 3A-3D: Labelling KO hLDLR K562 cells results in increased transduction by an scFv-trunc-VSVG pseudotyped virus. Labelled cells were generated by incubating hLDLR KO K562 cells at 37° C. for 30 minutes in 1 μM NHS 5′FAM in PBS, washing well before and after incubation. Standard lentivirus and scFv-truncVSVG pseudotyped viruses expressed BFP to evaluate transduction efficiency. Labelling success was monitored using an AF647-labelled anti-5′-FAM antibody, and by measuring 5′-FAM fluorescence, and the disappearance of 5′-FAM fluorescence was confirmed post-transduction, confirming transduction was facilitated by 5′FAM molecule. (FIG. 3A) WT K562 cells unlabeled and transduced with WT virus, (FIG. 3B) hLDLR KO K562 cells unlabeled and transduced with WT virus, (FIG. 3C) hLDLR KO K562 cells transduced with scFv-truncVSV-G pseudotyped virus, and (FIG. 3D) labelled hLDLR KO K562 cells transduced with scFv-truncated VSV-G pseudotyped virus.

FIG. 4: High speed concentration of scFv-truncVSVG pseudotyped lentivirus supernatant results in increased transduction efficiency. Methodology identical to FIG. 2, but 38 mL of virus supernatant was concentrated via high-speed centrifuge 15,600 rpm for 20 hrs at 4° C. and concentrated to 2 mL. Dilutions of concentrated virus were generated by diluting concentrated virus with medium.

FIG. 5: Sequences of fusion protein.

FIG. 6: Project Overview—cell surface engineering and viral pseudotyping.

FIG. 7: WT and KO LDL-R K562 cells were transduced with VSV-G lentivirus with or without 20 g/ml of polybrene. BFP expression was measured 48 hours post transduction using flow cytometry.

FIG. 8: WT K562 cells were incubated with 10 μg/ml I1 antibody with or without 20 μg/ml polybrene for 30 minutes on ice and BFP expression was measured using flow cytometry 48 hours post transduction.

FIG. 9: KO LDL-R K562 cells were incubated with various linker lengths indicated in the figure for 40 minutes at 37° C. and BFP expression was measured using flow cytometry 48 hours post transduction.

FIG. 10: KO LDL-R K562 cells were incubated with increasing concentrations of Maleimide-FITC, indicated in the figure, for 40 minutes at 37° C. and BFP level was measured using flow cytometry 48 hours post transduction.

FIG. 11: KO LDL-R K562 cells were incubated with either 1 μM NHS-FITC or 500 nM Amph-FITC^{5k} or 5 μM Maleimide-FITC or unlabeled for 40 minutes at 37° C. and BFP level was measured using flow cytometry 48 hours post transduction.

FIG. 12: Cell proliferation of NHS-FITC and Amph-FITC labeled cells with and without polybrene (20 μg/ml) and unlabeled cells with or without polybrene was calculated using a hemocytometer and trypan blue.

FIG. 13: KO LDL-R labeled and unlabeled K562 cells were incubated with 100 μM Methyl-Beta-Cyclodextrin clathrin inhibitor for 1 hr at 37° C., followed by transduction with aFITC/VSV-G lentivirus. BFP level was measured using flow cytometry, 48 hours post transduction.

FIG. 14: Virus binding is a better indicator of VSV-G LV gene delivery efficiency than LDLR expression. WT or hLDLR K562 cells were stained with virus for 30 min at room temperature, then stained with I1 antibody for 30 min on ice, followed by anti-mouse IgG antibody for 20 min on ice, then analyzed by flow cytometry. The same procedure was performed for all other mammalian cell lines indicated in the plots. The coefficient of determination (R²) was calculated in Prism.

FIG. 15: Surface modification improves VSV-G LV transduction of various cell lines and primary cells. Mammalian cell lines as indicated in the plot and pre-activated canine T cells were labeled with NHS-FITC (1 μM) for 40 minutes at 37° C., followed by virus transduction. BFP level was measured using flow cytometry 48 hours post-transduction.

FIG. 16: Naïve human T cell transduction and T cell phenotypes. Unactivated T cells (Donor 1) were pre-labeled with both FITC-conjugated anti-CD5 and anti-CD45 antibody followed by virus transduction in the presence of polybrene (10 ug/ml). Cells were maintained in the presence of hIL2 (10 ng/ml) and analyzed by flow cytometry 7 days post-transduction for CD19 CAR expression (APC channel) and T cell phenotypes (CD45RA-Alex488, CCR7-PE).

FIG. 17: Donor 2—Optimization of the labeling approach for naïve human T cell transduction. Unactivated T cells (Donor 2) were pre-labeled with NHC-FITC, FITC-conjugated anti-CD5, anti-CD45 antibody, anti-CD5/CD45 combo or anti-CD19, followed by virus transduction in the presence or absence of polybrene (10 ug/ml). Cells were maintained in the presence of hIL2 (10 ng/ml) and analyzed by flow cytometry 7 days post-transduction for CD19 CAR expression (APC channel). UNL: unlabeled, NTD: untransduced.

FIG. 18: Donor 2—Naïve human T cell transduction phenotyping. Transduced T cells from FIG. 17 were stained with CD45RA-Alex488 and CCR7-PE to assess the changes of T cell phenotypes after transduction. Activated T cells were included as control. Naïve T cell phenotype: CD45RA+CCR7+. CD45RA+CCR7+ naïve T cells were further analyzed for the CD19 CAR expression (APC channel).

### DESCRIPTION OF ILLUSTRATIVE EMBODIMENTS

Lentiviral transduction is a commonly used technology in modern laboratories and clinical therapies due to their safe and stable integration of genes into human cell genomes. The human Low Density Lipoprotein Receptor (hLDLR) has recently been identified as the natural target of VSV-G and serves as the entry receptor for VSV-G pseudotyped lentiviral vectors. However, many cell types have poor transducibility by the most widely used VSV-G pseudotyped lentiviral vectors, mostly due to the lack of expression of the proper lentiviral entry receptor, hLDLR. These include murine immune cell types, such as T cells, canine immune cells, and also human naïve T cells, certain epithelial cells, and more. The present studies aimed to overcome this limitation in these cell types with the design of a broad method to increase the efficiency of transduction. The studies here showed that in cells without hLDLR expression, chemical labelling resulted in increased lentiviral transduction ex vivo, using pseudotyped lentivirus.

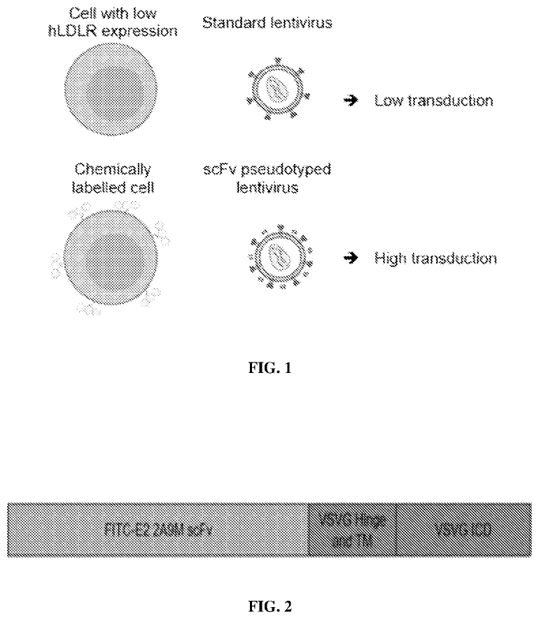

Thus, in certain embodiments, there is provided herein a strategy to chemically label cell surfaces with a small molecule, priming them for docking and lentivirus entry. These cells can then be efficiently transduced by pseudotyped lentiviruses that contain envelope proteins that target the cell-surface small molecule. This strategy dramatically improved lentiviral transduction efficiency (FIG. 1).

Further provided herein as methods for the use of the chemically labeled cells transduced with pseudotyped lentiviruses for gene therapy and lentivirus-based in vivo gene delivery, human naïve T cell engineering, and genetic screens using murine T cells for T cell immunology and cancer immunology research. The present methods and compositions can also be used to significantly accelerate the development of various disease models using lentivirus-based disease induction.

These and other aspects of the disclosure are described in detail below.

### I. Lentiviral Transduction

Existing methods of transducing murine T cells with VSV-G based lentivirus largely experience low transduction efficiency, ranging from 5-10%. In canine hematopoietic cells, there is also low transduction efficiency, typically less than 1%, and improvements in these cell types have only raised transduction to approximately 12%. In these cell types, and other non-transducible cell types, this is mostly due to the lack of an optimal entry receptor, hLDLR, for the lentivirus particles. Manipulation of primary T cells with specific culturing methods, which is the current method to improve transduction, to improve lentiviral transduction likely leads to undesired cell phenotypes.

In certain embodiments, the present methods provide a strategy of chemically labeling cells, by various methods, to offer a simple platform to achieve higher efficiency lentiviral transduction with high applicability and flexibility towards cell type. Chemical labelling makes use of easily purchased chemicals and does not alter cell genotype. Lentiviral pseudotyping overcomes the limitation of hLDLR entirely, providing methods for lentiviral-based transduction in animal cells such as murine and canine cells, as well as non-transducible human cell types such as naïve T cells.

The low-density lipoprotein receptor (LDL-R) is a mosaic protein of 839 amino acids (after removal of 21-amino acid signal peptide) that mediates the endocytosis of cholesterol-rich low-density lipoprotein (LDL). It is a cell-surface receptor that recognizes apolipoprotein B100 (ApoB100), which is embedded in the outer phospholipid layer of very low-density lipoprotein (VLDL), their remnants—i.e. intermediate-density lipoprotein (IDL), and LDL particles. The receptor also recognizes apolipoprotein E (ApoE) which is found in chylomicron remnants and IDL. In humans, the LDL receptor protein is encoded by the LDLR gene on chromosome 19. It belongs to the low-density lipoprotein receptor gene family. It is most significantly expressed in bronchial epithelial cells and adrenal gland and cortex tissue.

Disruption of LDL-R can lead to higher LDL-cholesterol as well as increasing the risk of related diseases. Individuals with disruptive mutations (defined as nonsense, splice site, or indel frameshift) in LDLR have an average LDL-cholesterol of 279 mg/dL, compared with 135 mg/dL for individuals with neither disruptive nor deleterious mutations. Disruptive mutations were 13 times more common in individuals with early-onset myocardial infarction or coronary artery disease than in individuals without either disease.

The human LDLR gene resides on chromosome 19 at the band 19p13.2 and is split into 18 exons. Exon 1 contains a signal sequence that localizes the receptor to the endoplasmic reticulum for transport to the cell surface. Beyond this, exons 2-6 code the ligand binding region; 7-14 code the epidermal growth factor (EGF) domain; 15 codes the oligosaccharide rich region; 16 (and some of 17) code the membrane spanning region; and 18 (with the rest of 17) code the cytosolic domain. This gene produces 6 isoforms through alternative splicing.

This protein belongs to the LDLR family and is made up of a number of functionally distinct domains, including 3 EGF-like domains, 7 LDL-R class A domains, and 6 LDL-R class B repeats. Representative sequences can be found at NP_000518 (protein) and NP_000527 (mRNA).

The N-terminal domain of the LDL receptor, which is responsible for ligand binding, is composed of seven sequence repeats (~50% identical). Each repeat, referred to as a class A repeat or LDL-A, contains roughly 40 amino acids, including 6 cysteine residues that form disulfide bonds within the repeat. Additionally, each repeat has highly conserved acidic residues which it uses to coordinate a single calcium ion in an octahedral lattice. Both the disulfide bonds and calcium coordination are necessary for the structural integrity of the domain during the receptor's repeated trips to the highly acidic interior of the endosome. The exact mechanism of interaction between the class A repeats and ligand (LDL) is unknown, but it is thought that the repeats act as “grabbers” to hold the LDL. Binding of ApoB requires repeats 2-7 while binding ApoE requires only repeat 5 (thought to be the ancestral repeat).

Next to the ligand binding domain is an EGF precursor homology domain (EGFP domain). This shows approximately 30% homology with the EGF precursor gene. There are three “growth factor” repeats; A, B and C. A and B are closely linked while C is separated by the YWTD repeat region, which adopts a beta-propeller conformation (LDL-R class B domain). It is thought that this region is responsible for the pH-dependent conformational shift that causes bound LDL to be released in the endosome.

A third domain of the protein is rich in O-linked oligosaccharides but appears to show little function. Knockout experiments have confirmed that no significant loss of activity occurs without this domain. It has been speculated that the domain may have ancestrally acted as a spacer to push the receptor beyond the extracellular matrix. The single transmembrane domain of 22 (mostly) non-polar residues crosses the plasma membrane in a single alpha helix. The cytosolic C-terminal domain contains ~50 amino acids, including a signal sequence important for localizing the receptors to clathrin-coated pits and for triggering receptor-mediated endocytosis after binding. Portions of the cytosolic sequence have been found in other lipoprotein receptors, as well as in more distant receptor relatives.

The non-transducible cells may be labeled with fluorescein or its derivatives, such as by a small molecule including but not limited to NHS-5′FAM, Maleimide-5′FAM, 1,2-Distearoyl-sn-glycero-3-phosphoethanolamine (DSPE)-Poly-ethylene-glycol(PEG)-Fluorescein, or by a bridge molecule, such as an FITC-conjugated antibody specific for a target cell surface protein. Different derivatives of Fluorescein, including FITC, 5′FAM, 6′FAM, that are recognized by an anti-FITC scFv can be used here for cell labeling. There can be no linker, rigid (e.g., hexanoic acid) or flexible linkers (e.g., PEG) of various lengths between the chemical group (e.g., NHS, Maleimide, DSPE), and fluorescein. The chemical group can also have a sulfo-modification which introduces a negative charge and limits the chemical labeling on cell surface proteins. The above molecules label cells through different labeling chemistries, for example, the NHS group reacts with the primary amine groups on protein molecules, the maleimide group reacts with free thiol groups on proteins, and DSPE directly insert in the lipid bilayer of cell membrane. Cells can be labeled with a single fluorescein-bearing molecule, or a combination of different fluorescein-bearing molecules.

Further provided herein are VSV-G chimeric proteins comprising a N-terminal single-chain antibody fragments (scFv) directed against fluorescein, wherein the VSV-G and the scFv may be separated by linker, such as a flexible linker. Also provided herein is a nucleic acid encoding the VSV-G chimeric protein and vector comprising the nucleic acid molecule of VSV-G chimeric fusion protein.

An exemplary nucleic acid encoding the vector pCAG-FITC E2-VSV is provided below. The present nucleic acid may have at least 80%, 85%, 90%, 95%, 96%, 97%, 98%, 99% or 100% sequence identity to SEQ ID NO:1:

| (pCAG-FITC E2-VSV) | |
|---|---|
| SEQ ID NO: 1 | |
| 1 | gtcgacattg attattgact agttattaat agtaatcaat tacggggtca ttagttcata |
| 61 | gcccatatat ggagttccgc gttacataac ttacggtaaa tggcccgcct ggctgaccgc |
| 121 | ccaacgaccc ccgcccattg acgtcaataa tgacgtatgt tcccatagta acgccaatag |
| 181 | ggactttcca ttgacgtcaa tgggtggact atttacggta aactgcccac ttggcagtac |
| 241 | atcaagtgta tcatatgcca agtacgcccc ctattgacgt caatgacggt aaatggcccg |
| 301 | cctggcatta tgcccagtac atgaccttat gggactttcc tacttggcag tacatctacg |
| 361 | tattagtcat cgctattacc atgggtcgag gtgagcccca cgttctgctt cactctcccc |
| 421 | atctcccccc cctccccacc cccaattttg tatttattta ttttttaatt attttgtgca |
| 481 | gcgatggggg cggggggggg gggggcgcgc gccaggcggg gcggggcggg gcgaggggcg |
| 541 | gggcggggcg aggcggagag gtgcggcggc agccaatcag agcggcgcgc tccgaaagtt |
| 601 | tccttttatg gcgaggcggc ggcggcggcg gccctataaa aagcgaagcg cgcggcgggc |
| 661 | gggagtcgct gcgttgcctt cgccccgtgc cccgctccgc gccgcctcgc gccgcccgcc |
| 721 | ccggctctga ctgaccgcgt tactcccaca ggtgagcggg cgggacggcc cttctcctcc |
| 781 | gggctgtaat tagcgcttgg tttaatgacg gctcgtttct tttctgtggc tgcgtgaaag |
| 841 | ccttaaaggg ctccgggagg gccctttgtg cgggggggag cggctcgggg ggtgcgtgcg |
| 901 | tgtgtgtgtg cgtggggagc gccgcgtgcg gcccgcgctg cccggcggct gtgagcgctg |
| 961 | cgggcgcggc gcggggcttt gtgcgctccg cgtgtgcgcg aggggagcgc ggccgggggc |
| 1021 | ggtgccccgc ggtgcggggg ggctgcgagg ggaacaaagg ctgcgtgcgg ggtgtgtgcg |
| 1081 | tgggggggtg agcagggggt gtgggcgcgg cggtcgggct gtaacccccc cctgcacccc |
| 1141 | cctccccgag ttgctgagca cggcccggct tcgggtgcgg ggctccgtgc ggggcgtggc |
| 1201 | gcggggctcg ccgtgccggg cggggggtgg cggcaggtgg gggtgccggg cggggcgggg |
| 1261 | ccgcctcggg ccggggaggg ctcgggggag gggcgcggcg gccccggagc gccggcggct |
| 1321 | gtcgaggcgc ggcgagccgc agccattgcc ttttatggta atcgtgcgag agggcgcagg |
| 1381 | gacttccttt gtcccaaatc tggcggagcc gaaatctggg aggcgccgcc gcaccccctc |
| 1441 | tagcgggcgc gggcgaagcg gtgcggcgcc ggcaggaagg aaatgggcgg ggagggcctt |
| 1501 | cgtgcgtcgc cgcgccgccg tccccttctc catctccagc ctcggggctg ccgcaggggg |
| 1561 | acggctgcct tcggggggga cggggcaggg cggggttcgg cttctggcgt gtgaccggcg |
| 1621 | gctctagagc ctctgctaac catgttcatg ccttcttctt tttcctacag ctcctgggca |
| 1681 | acgtgctggt tattgtgctg tctcatcatt ttggcaaaga attcgccgcc accatggaaa |
| 1741 | cagatacctt gcttctctgg gtgctgctct tgtgggtccc cggatcaaca ggggaacaga |
| 1801 | agctgatcag cgaggaggat ctgcaggttc agttggtaga aagcggtggc aatttggtcc |
| 1861 | agcctggcgg atctttgcgc ctttcctgtg ctgcatcagg ctttactttt gggtctttct |
| 1921 | caatgtcctg ggtaaggcaa gccccaggag gcggactcga gtgggtagcc ggactgtcag |
| 1981 | ctcgatcctc cctcactcat tatgccgact ccgttaaggg acggtttacc atctccaggg |
| 2041 | ataatgccaa aaatagcgtc tacctccaaa tgaactcttt gcgagtggaa gatacagccg |
| 2101 | tctactattg cgcccgcagg agctacgatt caagcggcta ttggggtcat ttctattcct |
| 2161 | acatggacgt ttggggccaa gggaccctcg tgaccgttag ctcaggaggg ggtggtagcg |
| 2221 | ggggaggagg gagtgggggc ggtggaagcc agtctgtgct cacccaacca tcttcagtaa |
| 2281 | gtgcagcacc agggcaaaag gtaactattt cctgtagtgg gtctacttcc aatataggta |
| 2341 | acaactacgt gtcatggtac cagcaacacc caggcaaagc acccaaattg atgatctacg |
| 2401 | acgtgtcaaa acgcccctca ggggtacctg atcgattttc aggttctaaa tctgggaata |
| 2461 | gtgcttcact tgatatctca ggtctgcaaa gcgaagatga agcagattac tactgcgctg |
| 2521 | catgggacga ttccttgtct gaatttcttt tcggaactgg caccaaactg accgtattgg |
| 2581 | ggaaaaatcc aatcgagctt gtagaaggtt ggttcagtag ttggaaaagc tctattgcct |
| 2641 | cttttttctt tatcataggg ttaatcattg gactattctt ggttctccga gttggtatcc |
| 2701 | atctttgcat taaattaaag cacaccaaga aaagacagat ttatacagac atagagatga |
| 2761 | accgacttgg aaagtaagcg gccgcactcc tcaggtgcag gctgcctatc agaaggtggt |
| 2821 | ggctggtgtg gccaatgccc tggctcacaa ataccactga gatctttttc cctctgccaa |
| 2881 | aaattatggg gacatcatga agccccttga gcatctgact tctggctaat aaaggaaatt |
| 2941 | tattttcatt gcaatagtgt gttggaattt tttgtgtctc tcactcggaa ggacatatgg |
| 301 | gagggcaaat catttaaaac atcagaatga gtatttggtt tagagtttgg caacatatgc |
| 3061 | catatgctgg ctgccatgaa caaaggtggc tataaagagg tcatcagtat atgaaacagc |
| 3121 | cccctgctgt ccattcctta ttccatagaa aagccttgac ttgaggttag atttttttta |
| 3181 | tattttgttt tgtgttattt ttttctttaa catccctaaa attttcctta catgttttac |
| 3241 | tagccagatt tttcctcctc tcctgactac tcccagtcat agctgtccct cttctcttat |
| 3301 | gaagatccct cgacctgcag cccaagcttg gcgtaatcat ggtcatagct gtttcctgtg |
| 3361 | tgaaattgtt atccgctcac aattccacac aacatacgag ccggaagcat aaagtgtaaa |
| 3421 | gcctggggtg cctaatgagt gagctaactc acattaattg cgttgcgctc actgcccgct |
| 3481 | ttccagtcgg gaaacctgtc gtgccagcgg atccgcatct caattagtca gcaaccatag |
| 3541 | tcccgcccct aactccgccc atcccgcccc taactccgcc cagttccgcc cattctccgc |
| 3601 | cccatggctg actaattttt tttatttatg cagaggccga ggccgcctcg gcctctgagc |
| 3661 | tattccagaa gtagtgagga ggcttttttg gaggcctagg cttttgcaaa aagctaactt |
| 3721 | gtttattgca gcttataatg gttacaaata aagcaatagc atcacaaatt tcacaaataa |
| 3781 | agcatttttt tcactgcatt ctagttgtgg tttgtccaaa ctcatcaatg tatcttatca |
| 3841 | tgtctggatc cgctgcatta atgaatcggc caacgcgcgg ggagaggcgg tttgcgtatt |
| 3901 | gggcgctctt ccgcttcctc gctcactgac tcgctgcgct cggtcgttcg gctgcggcga |
| 3961 | gcggtatcag ctcactcaaa ggcggtaata cggttatcca cagaatcagg ggataacgca |
| 4021 | ggaaagaaca tgtgagcaaa aggccagcaa aaggccagga accgtaaaaa ggccgcgttg |
| 4081 | ctggcgtttt tccataggct ccgcccccct gacgagcatc acaaaaatcg acgctcaagt |
| 4141 | cagaggtggc gaaacccgac aggactataa agataccagg cgtttccccc tggaagctcc |
| 4201 | ctcgtgcgct ctcctgttcc gaccctgccg cttaccggat acctgtccgc ctttctccct |
| 4261 | tcgggaagcg tggcgctttc tcaatgctca cgctgtaggt atctcagttc ggtgtaggtc |
| 4321 | gttcgctcca agctgggctg tgtgcacgaa ccccccgttc agcccgaccg ctgcgcctta |
| 4381 | tccggtaact atcgtcttga gtccaacccg gtaagacacg acttatcgcc actggcagca |
| 4441 | gccactggta acaggattag cagagcgagg tatgtaggcg gtgctacaga gttcttgaag |
| 4501 | tggtggccta actacggcta cactagaagg acagtatttg gtatctgcgc tctgctgaag |
| 4561 | ccagttacct tcggaaaaag agttggtagc tcttgatccg gcaaacaaac caccgctggt |
| 4621 | agcggtggtt tttttgtttg caagcagcag attacgcgca gaaaaaaagg atctcaagaa |
| 4681 | gatcctttga tcttttctac ggggtctgac gctcagtgga acgaaaactc acgttaaggg |
| 4741 | attttggtca tgagattatc aaaaaggatc ttcacctaga tccttttaaa ttaaaaatga |
| 4801 | agttttaaat caatctaaag tatatatgag taaacttggt ctgacagtta ccaatgctta |
| 4861 | atcagtgagg cacctatctc agcgatctgt ctatttcgtt catccatagt tgcctgactc |
| 4921 | cccgtcgtgt agataactac gatacgggag ggcttaccat ctggccccag tgctgcaatg |
| 4981 | ataccgcgag acccacgctc accggctcca gatttatcag caataaacca gccagccgga |
| 5041 | agggccgagc gcagaagtgg tcctgcaact ttatccgcct ccatccagtc tattaattgt |
| 5101 | tgccgggaag ctagagtaag tagttcgcca gttaatagtt tgcgcaacgt tgttgccatt |
| 5161 | gctacaggca tcgtggtgtc acgctcgtcg tttggtatgg cttcattcag ctccggttcc |
| 5221 | caacgatcaa ggcgagttac atgatccccc atgttgtgca aaaaagcggt tagctccttc |
| 5281 | ggtcctccga tcgttgtcag aagtaagttg gccgcagtgt tatcactcat ggttatggca |
| 5341 | gcactgcata attctcttac tgtcatgcca tccgtaagat gcttttctgt gactggtgag |
| 5401 | tactcaacca agtcattctg agaatagtgt atgcggcgac cgagttgctc ttgcccggcg |
| 5461 | tcaatacggg ataataccgc gccacatagc agaactttaa aagtgctcat cattggaaaa |
| 5521 | cgttcttcgg ggcgaaaact ctcaaggatc ttaccgctgt tgagatccag ttcgatgtaa |
| 5581 | cccactcgtg cacccaactg atcttcagca tcttttactt tcaccagcgt ttctgggtga |
| 5641 | gcaaaaacag gaaggcaaaa tgccgcaaaa aagggaataa gggcgacacg gaaatgttga |
| 5701 | atactcatac tcttcctttt tcaatattat tgaagcattt atcagggtta ttgtctcatg |
| 5761 | agcggataca tatttgaatg tatttagaaa aataaacaaa taggggttcc gcgcacattt |
| 5821 | ccccgaaaag tgccacctg |

An exemplary nucleic acid encoding the fusion protein is provided below. The present nucleic acids may have at least 80%, 85%, 90%, 95%, 96%, 97%, 98%, 99% or 100% sequence identity to SEQ ID NOs:2-7.

(SEQ ID NO: 2)
ATGGAAACAGATACCTTGCTTCTCTGGGTGCTGCTCTTGTGGGTCCCCGGA
TCAACAGGGGAACAGAAGCTGATCAGCGAGGAGGATCTGCAGGTTCAGTTGGTA
GAAAGCGGTGGCAATTTGGTCCAGCCTGGCGGATCTTTGCGCCTTTCCTGTGCTG
CATCAGGCTTTACTTTTGGGTCTTTCTCAATGTCCTGGGTAAGGCAAGCCCCAGG
AGGCGGACTCGAGTGGGTAGCCGGACTGTCAGCTCGATCCTCCCTCACTCATTAT
GCCGACTCCGTTAAGGGACGGTTTACCATCTCCAGGGATAATGCCAAAAATAGC
GTCTACCTCCAAATGAACTCTTTGCGAGTGGAAGATACAGCCGTCTACTATTGCG
CCCGCAGGAGCTACGATTCAAGCGGCTATTGGGGTCATTTCTATTCCTACATGGA
CGTTTGGGGCCAAGGGACCCTCGTGACCGTTAGCTCAGGAGGGGGTGGTAGCGG
GGGAGGAGGGAGTGGGGGCGGTGGAAGCCAGTCTGTGCTCACCCAACCATCTTC
AGTAAGTGCAGCACCAGGGCAAAAGGTAACTATTTCCTGTAGTGGGTCTACTTCC
AATATAGGTAACAACTACGTGTCATGGTACCAGCAACACCCAGGCAAAGCACCC
AAATTGATGATCTACGACGTGTCAAAACGCCCCTCAGGGGTACCTGATCGATTTT
CAGGTTCTAAATCTGGGAATAGTGCTTCACTTGATATCTCAGGTCTGCAAAGCGA
AGATGAAGCAGATTACTACTGCGCTGCATGGGACGATTCCTTGTCTGAATTTCTT
TTCGGAACTGGCACCAAACTGACCGTATTGGGGaaaaatccaatcgagcttgtagaaggttggttcagt
agttggaaaagctctattgcctcttttttctttatcatagggttaatcattggactattcttggttctccgagt
tggtatccatctttgcattaaattaaagcacaccaagaaaagacagatttatacagacatagagatgaaccgac
ttggaaagtaa
mIgK signal peptide:
(SEQ ID NO: 3)
ATGGAAACAGATACCTTGCTTCTCTGGGTGCTGCTCTTGTGGGTCCCCGGATCAA
CAGGG
Myc tag:
(SEQ ID NO: 4)
GAAGCTGATCAGCGAGGAGGATCTG
FITC-E2 scFv
(SEQ ID NO: 5)
CAGGTTCAGTTGGTAGAAAGCGGTGGCAATTTGGTCCAGCCTGGCGGATCTTTGC
GCCTTTCCTGTGCTGCATCAGGCTTTACTTTTGGGTCTTTCTCAATGTCCTGGGTA
AGGCAAGCCCCAGGAGGCGGACTCGAGTGGGTAGCCGGACTGTCAGCTCGATCC
TCCCTCACTCATTATGCCGACTCCGTTAAGGGACGGTTTACCATCTCCAGGGATA
ATGCCAAAAATAGCGTCTACCTCCAAATGAACTCTTTGCGAGTGGAAGATACAG
CCGTCTACTATTGCGCCCGCAGGAGCTACGATTCAAGCGGCTATTGGGGTCATTT
CTATTCCTACATGGACGTTTGGGGCCAAGGGACCCTCGTGACCGTTAGCTCAGGA
GGGGGTGGTAGCGGGGGAGGAGGGAGTGGGGGCGGTGGAAGCCAGTCTGTGCT
CACCCAACCATCTTCAGTAAGTGCAGCACCAGGGCAAAAGGTAACTATTTCCTGT
AGTGGGTCTACTTCCAATATAGGTAACAACTACGTGTCATGGTACCAGCAACACC
CAGGCAAAGCACCCAAATTGATGATCTACGACGTGTCAAAACGCCCCTCAGGGG
TACCTGATCGATTTTCAGGTTCTAAATCTGGGAATAGTGCTTCACTTGATATCTCA
GGTCTGCAAAGCGAAGATGAAGCAGATTACTACTGCGCTGCATGGGACGATTCC
TTGTCTGAATTTCTTTTCGGAACTGGCACCAAACTGACCGTATTGGGG:
VSVG-hinge:
(SEQ ID NO: 6)
aaaaatccaatcgagcttgtagaaggttggttcagtagttggaaaagctctattgcctct
VSVG-TM:
(SEQ ID NO: 7)
tttttctttatcatagggttaatcattggactattcttggttctc
An exemplary amino acid sequence of the fusion protein is provided below.
The present polypeptides may have at least 80%, 85%, 90%, 95%, 96%, 97%,
98%, 99% or 100% sequence identity to SEQ ID NOs: 8-14.
(SEQ ID NO: 8)
METDTLLLWVLLLWVPGSTGEQKLISEEDLQVQLVESGGNLVQPGGSLRLSC
AASGFTFGSFSMSWVRQAPGGGLEWVAGLSARSSLTHYADSVKGRFTISRDNAKNS
VYLQMNSLRVEDTAVYYCARRSYDSSGYWGHFYSYMDVWGQGTLVTVSSGGGGS
GGGGSGGGGSQSVLTQPSSVSAAPGQKVTISCSGSTSNIGNNYVSWYQQHPGKAPKL
MIYDVSKRPSGVPDRFSGSKSGNSASLDISGLQSEDEADYYCAAWDDSLSEFLFGTG
TKLTVLGKNPIELVEGWFSSWKSSIASFFFIIGLIIGLFLVLRVGIHLCIKLKHTKKRQIY
TDIEMNRLGK*
mIgK signal peptide:
(SEQ ID NO: 9)
METDTLLLWVLLLWVPGSTG
Myc tag:
(SEQ ID NO: 10)
EQKLISEEDL
FITC-E2 scFv:
(SEQ ID NO: 11)
QVQLVESGGNLVQPGGSLRLSCAASGFTFGSFSMSWVRQAPGGGLEWVAGLSARSS
LTHYADSVKGRFTISRDNAKNSVYLQMNSLRVEDTAVYYCARRSYDSSGYWGHFY
SYMDVWGQGTLVTVSSGGGGSGGGGSGGGGSQSVLTQPSSVSAAPGQKVTISCSGS
TSNIGNNYVSWYQQHPGKAPKLMIYDVSKRPSGVPDRFSGSKSGNSASLDISGLQSE
DEADYYCAAWDDSLSEFLFGTGTKLTVLG
VSVG-hinge:
(SEQ ID NO: 12)
KNPIELVEGWESSWKSSIAS
VSVG-TM:
(SEQ ID NO: 13)
FFFIIGLIIGLFLVL
VSVG-intracellular domain:
(SEQ ID NO: 14)
RVGIHLCIKLKHTKKRQIYTDIEMNRLGK

Nucleic acid molecules, which are also referred to herein as polynucleotides or nucleic acid sequences, include DNA, such as cDNA or genomic DNA, and RNA. It is understood that the term “RNA” as used herein comprises all forms of RNA including mRNA, tRNA and rRNA but also genomic RNA, such as in case of RNA of RNA viruses. In particular, embodiments reciting “RNA” are directed to mRNA. Further included are nucleic acid mimicking molecules known in the art such as synthetic or semi-synthetic derivatives of DNA or RNA and mixed polymers, both sense and antisense strands. They may contain additional non-natural or derivatized nucleotide bases, as will be readily appreciated by those skilled in the art. Such nucleic acid mimicking molecules or nucleic acid derivatives according to the present disclosure include peptide nucleic acid (PNA), phosphorothioate nucleic acid, phosphoramidate nucleic acid, 2′-O-methoxyethyl ribonucleic acid, morpholino nucleic acid, hexitol nucleic acid (HNA) and locked nucleic acid (LNA), an RNA derivative in which the ribose ring is constrained by a methylene linkage between the 2′-oxygen and the 4′-carbon (see, for example, Braasch and Corey, Chemistry & Biology 8, 1-7 (2001)). PNA is a synthetic DNA-mimic with an amide backbone in place of the sugar-phosphate backbone of DNA or RNA, as described by Nielsen er al., Science 254:1497 (1991); and Egholm et al., Nature 365:666 (1993).

The term “(poly)peptide” as used herein relates to polypeptides as well as peptides. The term “polypeptide”, as used herein interchangeably with the term “protein”, describes linear molecular chains of amino acids, including single chain proteins or their fragments, containing more than 30 amino acids, whereas the term “peptide” as used herein describes a group of molecules consisting of up to 30 amino acids. (Poly)peptides may further form oligomers consisting of at least two identical or different molecules. The corresponding higher order structures of such multimers are, correspondingly, termed homo- or heterodimers, homo- or heterotrimers etc. Such multimers also fall under the definition of the term “(poly)peptide”. The terms “polypeptide” and “peptide” also refer to naturally modified polypeptides/peptides where the modification results from, e.g., glycosylation, acetylation, phosphorylation and similar modifications which are well known in the art.

In particular, the present vector is a plasmid, cosmid, virus, bacteriophage or another vector used, e.g., conventionally in genetic engineering. The nucleic acid molecules provided herein may be inserted into several commercially available vectors suitable for the expression of eukaryotic proteins. Non-limiting examples include prokaryotic plasmid vectors, such as the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMClneo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen) and pCINeo (Promega).

The present nucleic acid molecules may also be inserted into vectors such that a translational fusion with another polynucleotide is generated. The other polynucleotide may encode a protein which may, e.g., increase the solubility and/or facilitate the purification of the chimeric protein. Non-limiting examples include pET32, pET41, pET43.

For vector modification techniques, see Sambrook and Russell “Molecular Cloning, A Laboratory Manual”, Cold Spring Harbor Laboratory, N.Y. (2001). Generally, vectors can contain one or more origins of replication (ori) and inheritance systems for cloning or expression, one or more markers for selection in the host, e.g., antibiotic resistance, and one or more expression cassettes. Suitable origins of replication (ori) include, for example, the Col E1, the SV40 viral and the M 13 origins of replication.

The coding sequences inserted in the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression of the coding sequences are well known to those skilled in the art. These elements comprise regulatory sequences ensuring the initiation of the transcription (e.g., translation initiation codon, promoters, enhancers, and/or insulators), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers, and/or naturally associated or heterologous promoter regions. In particular, the nucleic acid molecule of the present disclosure is operatively linked to such expression control sequences allowing its expression. The vector may further comprise nucleotide sequences encoding secretion signals as further regulatory elements. Such sequences are well known to persons skilled in the art. Furthermore, depending on the expression system used, leader sequences capable of directing the expressed polypeptide to a cellular compartment may be added to the coding sequence of the polynucleotide of the present disclosure. Such leader sequences are well known in the art.

Possible examples for regulatory elements ensuring the initiation of transcription comprise the cytomegalovirus (CMV) promoter, SV40-promoter, RSV-promoter (Rous sarcome virus), the lacZ promoter, the gai10 promoter, human elongation factor 1α-promoter, CMV enhancer, CaM-kinase promoter, the Autographa californica multiple nuclear polyhedrosis virus (AcMNPV) polyhedral promoter or the SV40-enhancer. For the expression in prokaryotes, a multitude of promoters including, for example, the tac-lac-promoter, the lacUV5 or the trp promoter, has been described. Examples for further regulatory elements in prokaryotes and eukaryotic cells comprise transcription termination signals, such as SV40-poly-A site or the tk-poly-A site or the SV40, lacZ and AcMNPV polyhedral polyadenylation signals, downstream of the polynucleotide.

Furthermore, the present vectors may comprise a selectable marker. Examples of selectable markers include neomycin, ampicillin and hygromycin resistance and the like. Specifically designed vectors allow the shuttling of DNA between different hosts, such as bacteria-fungal cells or bacteria-animal cells.

An expression vector as used herein is capable of directing the replication, and the expression, of the nucleic acid molecule and encoded VSV-G chimeric protein. Suitable expression vectors which comprise the described regulatory elements are known in the art such as pGreenPuro (System Biosciences, Mountain View, Calif., USA), pRc/CMV, pcDNA1, pcDNA3 (In-Vitrogene, as used, inter alia in the appended examples), pSPORT1 (GIBCO BRL) or pGEMHE (Promega), or prokaryotic expression vectors, such as lambda gt11, pJOE, the pBR1-MCS-series.

The nucleic acid molecules of the present disclosure as described herein above may be designed for direct introduction or for introduction via liposomes, phage vectors or viral vectors (e.g., adenoviral, retroviral) into the cell. Additionally, baculoviral systems or systems based on Vaccinia Virus or Semliki Forest Virus can be used as eukaryotic expression system for the nucleic acid molecules of the present disclosure.

The present disclosure further relates to a host cell comprising the nucleic acid molecule or the vector provided herein. Suitable prokaryotic hosts comprise, e.g., bacteria of the species Escherichia, Streptomyces, Salmonella or Bacillus. Suitable eukaryotic host cells are, e.g., yeasts such as Saccharomyces cerevisiae, Pichia pastoris, Schizosaccharomyces pombe or chicken cells, such as, e.g., DT40 cells. Insect cells suitable for expression are, e.g., Drosophila S2, Drosophila Kc, or Spodoptera Sf9 and Sf21 cells. Suitable zebrafish cell lines include, without being limiting, ZFL, SJD or ZF4.

Mammalian host cells that could be used include, human Hela, HEK293, HEK293T, H9 and Jurkat cells, mouse NIH3T3 and C127 cells, COS 1, COS 7 and CV1, quail QCI-3 cells, mouse L cells, mouse sarcoma cells, Bowes melanoma cells, human CAP or CAP-T cells and Chinese hamster ovary (CHO) cells. Also within the scope of the present disclosure are primary mammalian cells or cell lines. Primary cells are cells which are directly obtained from an organism. Suitable primary cells are, for example, mouse embryonic fibroblasts (MEF), mouse primary hepatocytes, cardiomyocytes and neuronal cells as well as mouse muscle stem cells (satellite cells), human dermal and pulmonary fibroblasts, human epithelial cells (nasal, tracheal, renal, placental, intestinal, bronchial epithelial cells), human secretory cells (from salivary, sebaceous and sweat glands), human endocrine cells (thyroid cells), human adipose cells, human smooth muscle cells, human skeletal muscle cells, and stable, immortalized cell lines derived thereof (for example hTERT or oncogene immortalized cells).

The host cell in accordance with this embodiment may for example be employed in methods for the amplification of vectors of the present disclosure, for the production of the fusion protein of the present disclosure or for the direct production of lentivirus particles, as described in more detail herein below.

The present disclosure further relates to a polypeptide encoded by the nucleic acid molecule provided herein. In further embodiment, there is provided a method of producing the VSV-G chimeric protein, the method comprising culturing the host cell under suitable conditions and isolating the produced VSV-G fusion protein.

Suitable conditions for culturing a prokaryotic or eukaryotic host are well known to the person skilled in the art. For example, suitable conditions for culturing bacteria are growing them under aeration in Luria Bertani (LB) medium. To increase the yield and the solubility of the expression product, the medium can be buffered or supplemented with suitable additives known to enhance or facilitate both. E. coli can be cultured from 4 to about 37° C., the exact temperature or sequence of temperatures depends on the molecule to be over-expressed. In general, the skilled person is also aware that these conditions may have to be adapted to the needs of the host and the requirements of the protein expressed. In case an inducible promoter controls the nucleic acid of the present disclosure in the vector present in the host cell, expression of the polypeptide can be induced by addition of an appropriate inducing agent. Suitable expression protocols and strategies are known to the skilled person.

Depending on the cell type and its specific requirements, mammalian cell culture can, e.g., be carried out in RPMI, Williams' E or DMEM medium containing 10% (v/v) FCS, 2 mM L-glutamine and 100 U/ml penicillin/streptomycine. The cells can be kept, e.g., at 37° C. or at 41° C. for DT40 chicken cells, in a 5% CO₂, water saturated atmosphere. Suitable media for insect cell culture is, e.g., TNM+10% FCS or SF900 medium. Insect cells are usually grown at 27° C. as adhesion or suspension culture. Suitable expression protocols for eukaryotic or vertebrate cells are well known to the skilled person and can be retrieved, e.g., from Sambrook and Russel, loc. cit.

The term “isolating” refers to a selective accumulation of the produced VSV-G chimeric protein, by removing the produced VSV-G chimeric protein from the host cells or from the medium in which the host cells have been cultured. In particular, the isolated VSV-G chimeric protein is 100% pure, i.e., is free of any other components that are not the VSV-G chimeric protein of the present disclosure. Methods of isolation of the chimeric protein produced are well-known in the art and comprise, without limitation, method steps such as ion exchange chromatography, gel filtration chromatography (size exclusion chromatography), affinity chromatography, high pressure liquid chromatography (HPLC), reversed phase HPLC, disc gel electrophoresis or immunoprecipitation, see, for example, in Sambrook and Russel, loc. cit.

The lentiviral vector particle may be pseudotyped with (a) a VSV-G chimeric protein domain encoded by the nucleic acid molecule of the present disclosure; and (b) a VSV-G not linked to a (poly)peptide comprising or consisting of a cell membrane-binding domain.

A “lentiviral vector particle”, also referred to herein as a “lentiviral vector”, is a vector based on a lentivirus virion, i.e., a subclass of retroviruses that can integrate into the genome of non-dividing target cells. A unique feature of lentiviruses is that they have a self-inactivated (SIN) region of replication in contrast to other retroviral vectors. Lentiviruses are well known in the art and have been described in detail, e.g., in Retroviruses, Coffin J M, Hughes S H, Varmus H E, Cold Spring Harbor (N.Y.): Cold Spring Harbour Laboratory Press; 1997; ISBN-10:0-87969-571-4; O'Connell R M, Balazs A B, Rao D S, Kivork C, Yang L, Baltimore D. Lentiviral vector delivery of human interleukin-7 (hIL-7) to human immune system (HIS) mice expands T lymphocyte populations. PLoS One. 2010 Aug. 6; 5(8):e12009; Mdtrai J, Chuah M K, VandenDriessche T. Recent advances in lentiviral vector development and applications. Mol Ther. 2010 March; 18(3):477-90.

A lentiviral vector particle can be based, e.g., on a lentivirus of the group of bovine, equine, feline, ovine/caprine or primate lentiviruses. In particular, the lentiviral vector is based on a primate lentivirus such as HIV1, HIV2 or SIV virus. Most preferred, the lentiviral vector is based on an HIV1 lentivirus. As the skilled person is aware, most (commercially available) lentiviral vectors represent a mixture of viral constituents from different viruses and are, hence, to some extent “hybrid” vectors. For example, a lentiviral vector may comprise constituents from HIV1, VSVg, CMV, WPRE viruses. Such hybrid vectors are explicitly envisaged in accordance with the present disclosure.

The term “pseudotyped”, as used herein in the context of viral vectors, refers to the modulation of the cell type specificity of a viral vector by integration of foreign viral envelope proteins. This approach is well known in the art and has been described for example in Bischof et al. (Flexibility in cell targeting by pseudotyping lentiviral vectors. Methods Mol Biol. 2010; 614:53-68). Using this approach, host tropism can be altered and/or the stability of the virus can be decreased or increased. For example, the use of VSV-G for pseudotyping a lentiviral virus has been described, e.g., in Burns et al. (Vesicular stomatitis virus G glycoprotein pseudotyped retroviral vectors: concentration to very high titer and efficient gene transfer into mammalian and nonmammalian cells. Proc Natl Acad Sci USA. 1993; 90(17): 8033-8037).

The term “a VSV-G not linked to a (poly)peptide comprising or consisting of a cell membrane-binding domain” is also referred to herein as wild type VSV-G. In this context, the term “wild type” only refers to the fact that no cell membrane-binding domain is fused to the VSV-G employed. However, the use of non-naturally occurring (i.e., modified) VSV-G molecules is not excluded, as long as they are not linked to a (poly)peptide comprising or consisting of a cell membrane-binding domain.

Further embodiments provide a lentivirus vector particle that is pseudotyped with two different types of VSV-G proteins, namely with the present VSV-G chimeric protein and with a VSV-G that has not been fused to a cell membrane-binding domain, i.e., a wild-type (wt) VSV-G (option (b)). In other words, each individual lentivirus vector particle expresses both modified and wild type VSV-G glycoproteins on its surface.

The present disclosure further relates to a method of producing the pseudotyped lentiviral vector particle, the method comprising transfecting into a host cell (i) one or more packaging plasmids encoding the virion proteins and accessory proteins needed for efficient production and packaging of the LTR-containing nucleic acid; (ii) a vector comprising the nucleic acid molecule of the present disclosure; and (iii) a vector comprising a nucleic acid molecule encoding a VSV-G not linked to a (poly)peptide comprising or consisting of a cell membrane-binding domain.

Such methods of producing pseudotyped lentiviral vectors are well known in the art and have been described, e.g., in Naldini, L. (1998). Host cells, in particular HEK 293, HEK293T, CAP or CAP-T cells are employed and a number of vectors, including the packaging vector(s) encoding the viral proteins, such as e.g. the capsid and the reverse transcriptase, as well as vectors carrying the nucleic acid molecules to be additionally introduced into the pseudotyped lentiviral vector particle are transfected or electroporated into these cells, or nucleofection is used to transfer said vectors. In addition, further vectors containing the genetic material to be delivered by the pseudotyped lentiviral vector particle may be transfected.

These vectors may be introduced into the host cells by direct introduction or by introduction via electroporation (using for example Multiporator (Eppendorf), Genepulser (BioRad), MaxCyte Transfection Systems (Maxcyte)), PEI (Polysciences Inc. Warrington, Eppelheim), Ca²⁺-mediated transfection or via liposomes (for example: “Lipofectamine” (Invitrogen)), non-liposomal compounds (for example: “Fugene” (Roche) or nucleofection (Lonza)) into cells.

The present disclosure further relates to a method for transducing cells, the method comprising the step of contacting cells to be transduced with the pseudotyped lentiviral vector particle under conditions suitable for transduction, thereby transducing said cells.

The term “transducing”, as used herein, is well known in the art and refers to the process of introducing genetic material into a cell and, optionally, its subsequent integration into the genome of said cell via viral vector particles. Said genetic material comprises or consists of viral RNA combined with one or more target RNA sequences (hereinafter referred to as target sequences) comprised in said viral vector particles intended for integration into the genome of a target cell.

The term “contacting” as used herein in the context of this method of the present disclosure refers to bringing the cells to be transduced (also referred to herein as “target cells”) into contact with a retroviral vector so that the transduction event can occur. Conditions for contacting that allow the transduction event to occur are well known in the art and may depend to a certain extent on the cell to be transduced. For example, some target cells are more difficult to transfect than other cells and may need to be transitioned into a specific culture medium before transduction with a viral vector can be achieved. Corresponding methods and conditions are described for example in Jacome et al. (Lentiviral-mediated Genetic Correction of Hematopoietic and Mesenchymal Progenitor Cells from Fanconi Anemia Patients. Mol Ther. 2009 June; 17(6): 1083-1092), Chu et al. (Efficient and Stable Gene Expression into Human Osteoclasts Using an HIV-1-Based Lentiviral Vector. DNA Cell Biol. 2008 June; 27(6): 315-320), or Poczobutt et al. (Benign mammary epithelial cells enhance the transformed phenotype of human breast cancer cells. BMC Cancer. 2010; 10: 373).

The cells to be transduced can be any cells of interest that are to be targeted for transduction with a viral vector, particularly non-transducible cells, such as with low LDLR. The term “cell/cells” as used herein can refer to single and/or isolated cells or to cells that are part of a multicellular entity such as a tissue, an organism or a cell culture. In other words the method can be performed in vivo, ex vivo or in vitro. In particular, the cells to be transduced are eukaryotic cells including any cell of a multi-cellular eukaryotic organism, more specifically cells from animals like vertebrates. More particularly, the cells to be transduced are mammalian cell. Depending on the particular goal to be achieved through modifying the genome of a mammalian cell by transducing it according to the method of the present disclosure, cells of different mammalian subclasses such as prototheria or theria may be used. For example, within the subclass of theria, in particular cells of animals of the infraclass eutheria, more particularly of the order primates, artiodactyla, perissodactyla, rodentia and lagomorpha are used in the method of the present disclosure. Furthermore, within a species one may choose a cell to be used in the method of the present disclosure based on the tissue type and/or capacity to differentiate equally depending on the goal to be achieved by modifying the genome via transducing a target cell according to the method of the present disclosure. Three basic categories of cells, which in principle can be transduced with the method of the present disclosure, make up the mammalian body: germ cells, somatic cells and stem cells. A germ cell is a cell that gives rise to gametes and thus is continuous through the generations. Stem cells can divide and differentiate into diverse specialized cell types as well as self-renew to produce more stem cells. In mammals there are two main types of stem cells: embryonic stem cells and adult stem cells. Somatic cells include all cells that are not gametes, gametocytes or undifferentiated stem cells. The cells of a mammal can also be grouped by their ability to differentiate. A totipotent (also known as omnipotent) cell is a cell that is able to differentiate into all cell types of an adult organism including placental tissue such as a zygote (fertilized oocyte) and subsequent blastomeres, whereas pluripotent cells, such as embryonic stem cells, cannot contribute to extraembryonic tissue such as the placenta, but have the potential to differentiate into any of the three germ layers endoderm, mesoderm and ectoderm. Multipotent progenitor cells have the potential to give rise to cells from multiple, but limited number of cell lineages. Further, there are oligopotent cells that can develop into only a few cell types and unipotent cells (also sometimes termed a precursor cell) that can develop into only one cell type. There are four basic types of tissues: muscle tissue, nervous tissue, connective tissue and epithelial tissue that cells to be used in the method of the present disclosure can be derived from, such as for example lymphoid lineage cells or neuronal stem cells.

The term “lymphoid lineage cells” refers to cells that are involved in the generation of lymphocytes and lymphocytes per se. The term “lymphocyte” refers to small lymphocytes (B and T lymphocytes, plasma cells) and natural killer cells as well-known in the art. Lymphoid lineage cells further include, e.g., lymphoid dendritic cells, as well as lymphocyte progenitor cells such as pro-lymphocytes, lymphoblasts, common lymphoid progenitor cells.

The term “epithelial cell” is well known in the art. Epithelial cells line cavities and surfaces of structures throughout the body and also form many glands. Epithelial tissues can be classified into simple epithelium (one cell thick) and stratified epithelium (several layers of cells). Epithelial cells are furthermore classified by their morphology into squamous, cuboidal, columnar and pseudostratified epithelial cells. For example, the human stomach and intestine is lined with epithelial cells. Further, epithelial cell lines include also breast carcinoma cells (such as, e.g., MCF7, MDA-MB-361 and T47D cells) or cells of the cell line HEK293T.

The term “adjuvant”, as used herein, relates to a compound that enhances the efficiency of the lentiviral transduction. Non-limiting examples of adjuvants include e.g. a poloxamer having a molecular weight of 12.8 kDa to about 15 kDa and polybrene.

The term “poloxamer” is well known in the art and refers to a non-ionic triblock copolymer composed of a central hydrophobic chain of polyoxypropylene flanked by two hydrophilic chains of polyoxyethylene.

The lentiviral vector particles and the adjuvant can be added simultaneously, e.g. as a mixture, to the target cells or in sequential mode, as long as both compounds are simultaneously in contact with the target cell to allow transduction.

### II. Methods of Use

The present disclosure provides methods of providing gene therapy and lentivirus-based in vitro gene delivery, human naïve T cell engineering, and genetic screens using murine T cells for T cell immunology and cancer immunology research.

In certain embodiments, the present disclosure provides methods of providing gene therapy for non-transducible cells. Gene therapy can be performed by gene transfer, gene editing (e.g., CRISPR), exon skipping, RNA-interference, trans-splicing or any other genetic modification of any coding or regulatory sequences in the cell, including those included in the nucleus, mitochondria or as commensal DNA (viral sequences contained in cells).

The two main types of gene therapy are the following: a therapy aiming the replacement of a deficient/abnormal gene: this is replacement gene therapy; or a therapy aiming gene editing: in such a case, the purpose is to provide to a cell the necessary tools so that the gene of interest is expressed: this is gene editing therapy.

In replacement gene therapy, the gene of interest may be a correct version of a gene which is deficient or mutated in a patient, as is the case for example in a genetic disease. In such a case, the gene of interest will restore the expression of the deficient or mutated gene. Of particular interest are deficient or mutated genes in patients exhibiting a disease which, once corrected in immune cells, in particular in B cells, T cells, monocytes or dendritic cells, more particular in B cells, improve the patient's disease or symptoms.

In gene therapy, it might be possible to use the present pseudotyped lentiviral particles and chemically labeled cells in therapy for immune cell engineering, in particular T or B cell engineering, by transducing said immune cells. It might be possible to generate regulatory B cells by expressing immunosuppressive proteins in B cells, for instance IL-10, or to induce the production of immunoregulatory proteins such as antibodies or fusion proteins.

It could also be possible to insert sequences favoring gene splicing, expression or regulation or gene editing. Tools such as CRISPR/Cas9 may be used for this purpose. This could be used to modify gene expression in T or B cells, in the case of autoimmunity or cancer, or to perturb the cycle of viruses in B cells. In such cases, in particular, the heterologous gene of interest is chosen from gRNA, nucleases, DNA templates and RNAi components, such as shRNA.

An immune cell is a cell involved in the immune system. It comprises notably B cells, T cells, NK cells, macrophages and dendritic cells. A B cell (or B lymphocyte) is an immune cell responsible for the production of antibodies and involved in the humoral immune response. A dendritic cell is an immune cell, which is accessory, i.e., it is an antigen-presenting cell. Its main function is to process the antigen material and present it on its cell surface to the T cells. A T cell (or T lymphocyte) is an immune cell involved in cell-mediated immune response. It may be chosen from killer T cells, helper T cells and gamma delta T cells.

The term “subject” or “patient” as used herein refers to any individual to which the subject methods are performed. Generally, the patient is human, although as will be appreciated by those skilled in the art, the patient may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

### III. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Generation and Characterization of Chemically Labeled Cells

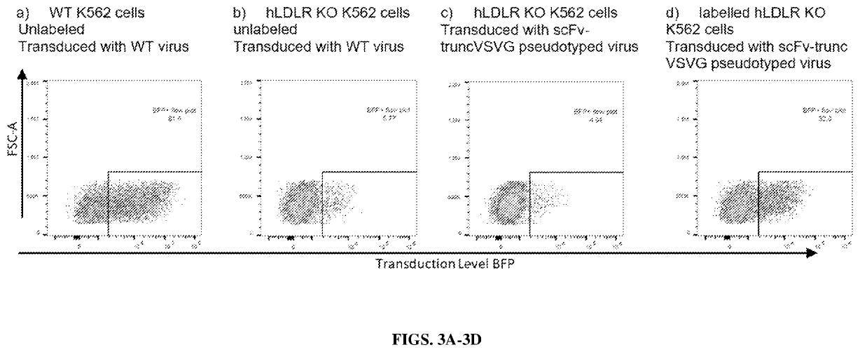

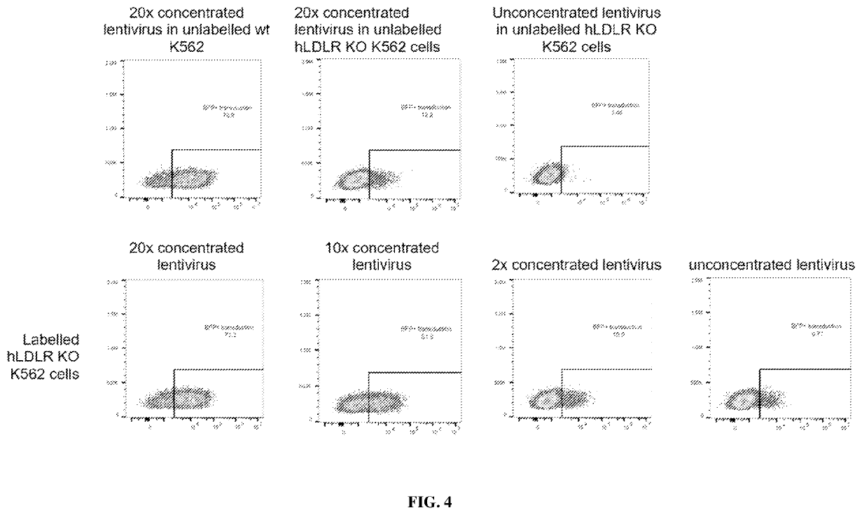

First, a K562 cell line was developed with hLDLR knocked out via CRISPR-Cas9 to model various cell types with low hLDLR expression. The CRISPR Cas9 cassette targeting the hLDLR gene locus was transduced into K562 cells via lentiviral vector. After 6 days of selection by puromycin, the transduction efficiency of the hLDLR KO cells using BFP-expressing standard VSV-G pseudotyped lentivirus. In these hLDLR KO cells, transduction by VSV-G pseudotyped lentivirus is only 7% (FIG. 3B), from a typical transduction efficiency of about 60% (FIG. 3A). This shows that transduction efficiency correlates with the presence of the hLDLR in K562 cells, confirming that a lack of hLDLR results in a lower transduction.

The hLDLR KO K562 cells were then chemically labelled with NHS-5′FAM at 1 μM and transduced with a pseudotyped lentivirus expressing BFP. NHS 5′FAM is a fluorescein molecule conjugated to N-hydroxysuccinimide at the 5′ carbon, allowing it to covalently form amide bonds with free primary amines. This lentivirus contained both the standard VSV-G envelope and a chimeric protein scFv-truncVSVG, produced by transfection of HEK293T cells, using a 1:1:0.25:0.25 plasmid mass ratio of sPAX:BFP:MD2G:scFv-truncVSVG. scFv-truncVSVG is composed of the VSV-G intracellular domain and transmembrane domain fused to an N-terminus domain of an anti-fluorescein scFv, 2A9M derived from FITC-E2 (FIG. 2). This clone targets a region of FITC that is similar to 5′FAM, allowing it to be used. Alternative anti-FITC scFvs such as 4m5.3, 4-4-20 (and variants) can be used here for generating the scFv-truncVSVG as well. The transmembrane domain and intracellular domain could also be derived from proteins with minimal intracellular domain such as CD8-alpha, PDGFR, and MHC-class I. The ratio of the plasmids for lentivirus preparation could be empirically determined. The lentivirus vector can contain a fluorescent protein reporter (e.g., BFP, RFP), a chimeric antigen receptor (CAR), a therapeutic cytokine (e.g., IL2, IL12), a guide-RNA for CRISPR-based genome editing, or any other desired protein or non-protein-encoding gene. Lentivirus can be used as crude culture supernatant, affinity purified, or concentrated using approaches such as ultracentrifugation.

Transduction was performed by adding equivalent volumes of cells at 1e6 cells/mL and lentiviral supernatant collected from HEK293T cells at 60 hrs post-transfection. Polybrene (10 mg/ml) was added at 1000× dilution and the viral-cell mix was centrifuged at 2000 g for 1.5 hrs at 35° C. Transduction of labelled hLDLR KO K562 cells with scFv-truncVSVG pseudotyped viruses resulted in a significant increase from 4% to 32% transduction efficiency (FIGS. 3C-3D). This showed that the strategy of chemical labelling and transduction with pseudotyped lentivirus resulted in increased transduction efficiency in cells with low expression of hLDLR.

After the initial successful transduction of labelled cells by scFv-truncVSVG pseudotyped lentivirus, studies were performed to raise the transduction efficiency by concentrating the raw viral supernatant. It was hypothesized that due to the need to incorporate both VSVG, scFv-truncVSVG, Gag/Pol, and BFP plasmid into a single functional viral particle, there was a low probability of assembling functional viral particles in HEK293T cells post-transfection. This would result in a low viral titer. The raw viral supernatant collected from HEK293T cells 60 hrs post-transfection was centrifuged at 15,600 rpm for 20 hrs at 4° C. and resulting in a 20-fold concentrated viral supernatant and raising virus titer 20-fold.

Upon 20-hour high-speed centrifugation, the lentiviral transduction efficiency increased to 72% (FIG. 4, bottom left), equivalent to transduction of wt K562 cells with standard lentivirus. (FIG. 3A). As expected in the control setup, transduction of the unlabeled WT K562 cells or hLDLR KO with concentrated virus was slightly increased (FIG. 4, upper panels), likely due to the non-specific and inefficient cell-intrinsic micropinocytosis. Furthermore, dilutions of the concentrated viral supernatant resulted in corresponding drops in transduction efficiency (FIG. 4, bottom panels), proving that raised transduction was due to an increase in viral particle concentration. In conclusion, a strategy was developed that facilitates transduction of cells with low hLDLR expressing cells to an equivalent level of efficiency as optimal transduction efficiencies in hLDLR expressing cells.

### Example 2—Transgene Delivery to Cells

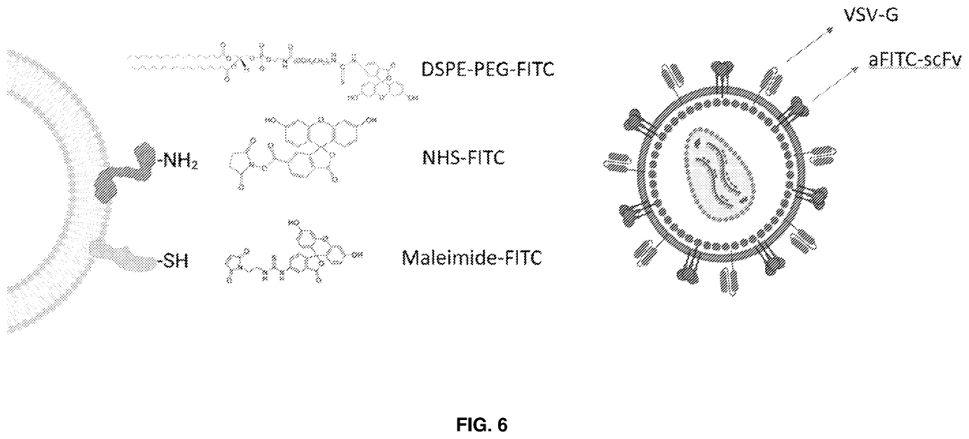

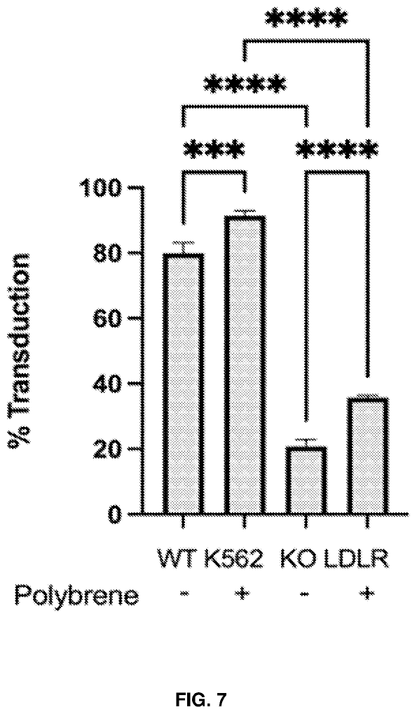

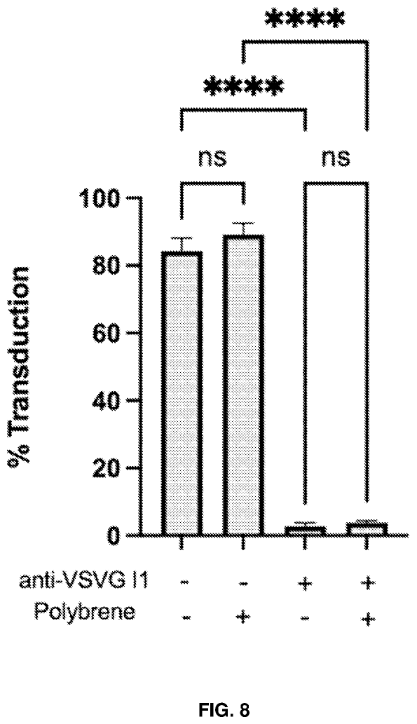

Based on the above results, the inventors hypothesized that scFv-truncVSVG pseudotyped viruses could deliver a transgene efficiently to mammalian cells conjugated with FITC molecules via a chemical approach, including NHS-chemistry (NHS-FITC/5′FAM), lipid insertion (DSPE-PEG-FITC, 1,2-distearoyl-sn-glycero-3-phosphoethanolamine-N-[poly(ethylene glycol)2000-N′-carboxyfluorescein](ammonium salt)), and maleimide chemistry (Mal-FITC/5′FAM) (FIG. 6). They first confirmed that polybrene, a standard cationic polymer widely used for promoting viral transduction, failed to restore virus transduction of hLDLR-KO K562 cells (FIG. 7). Notably, blocking VSV-G on the virus surface using an anti-VSV-G antibody (clone I1) completely abolished virus transduction regardless of the presence of polybrene (FIG. 8).

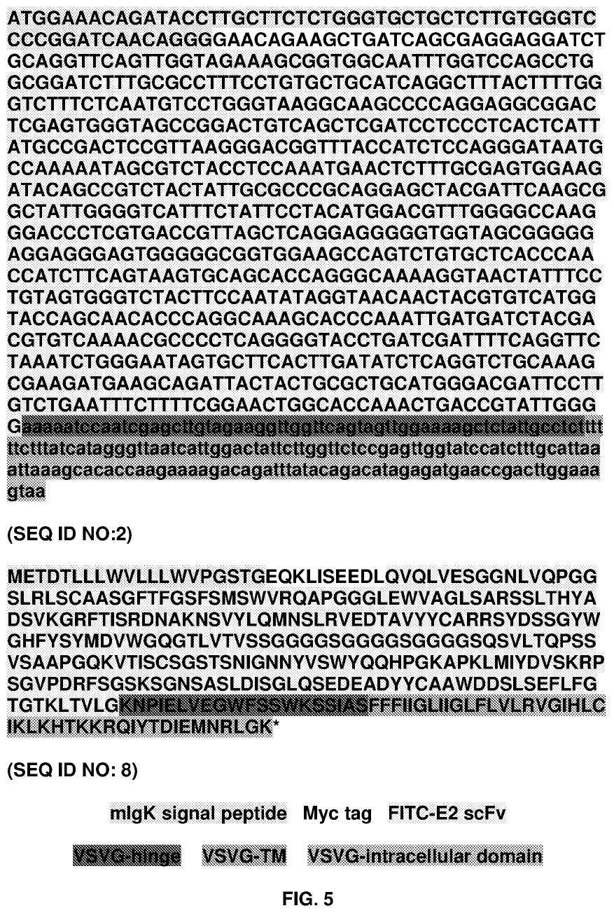

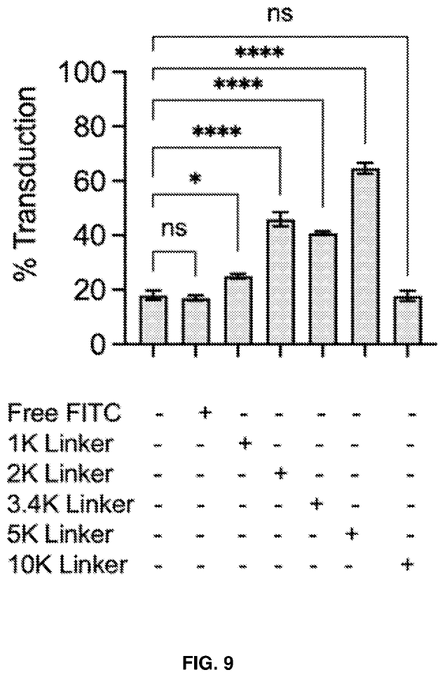

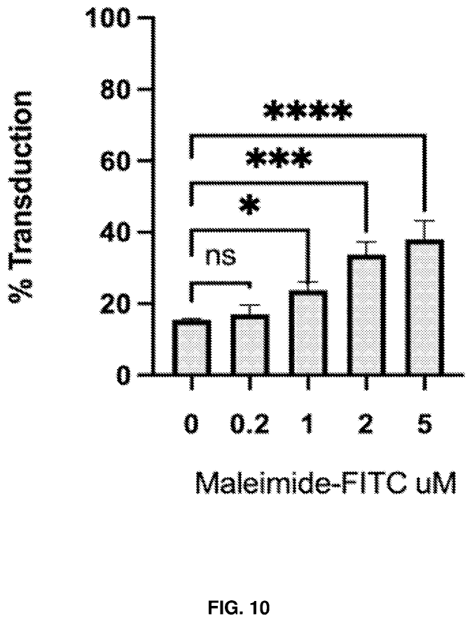

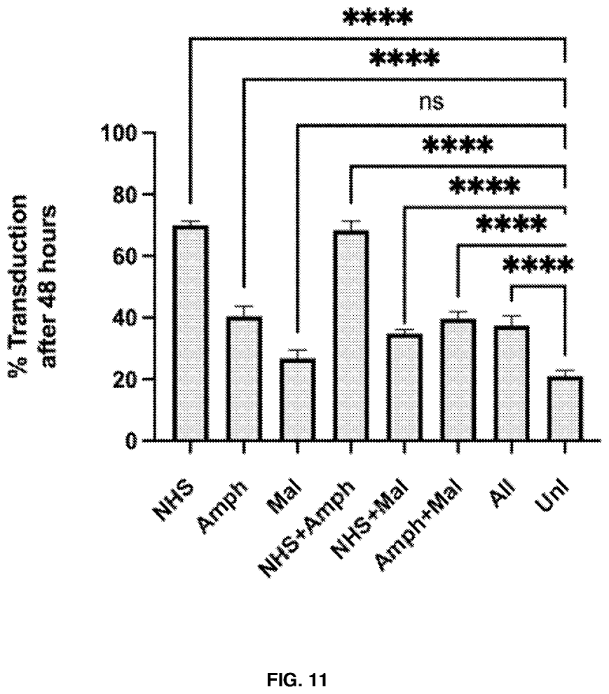

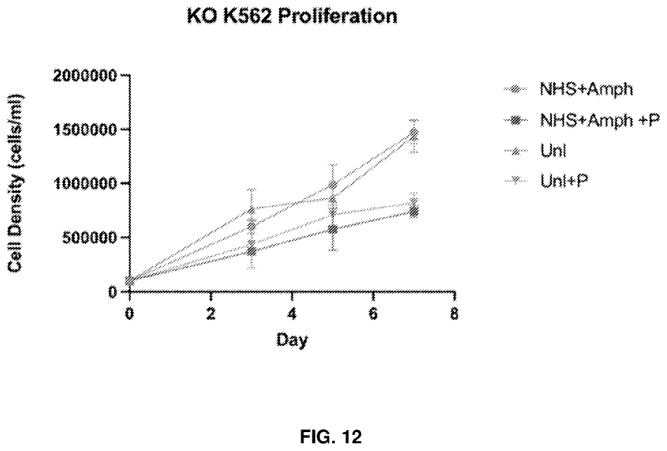

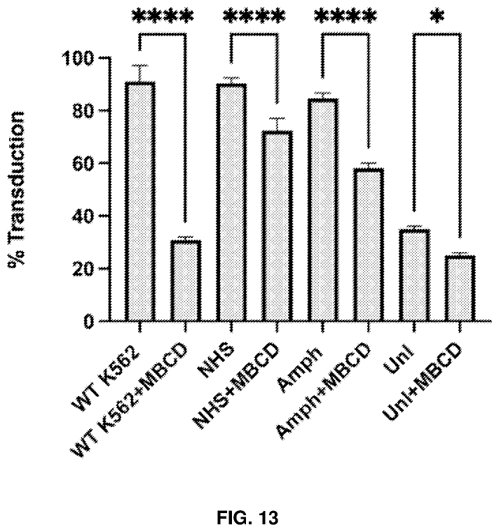

The inventors carried out systemic optimization of the chemical approach for labeling mammalian cells with FITC. For the lipid insertion approach, they assessed the impact of the PEG linker length and found that DSPE-PEG-FITC with a PEG5k linker performs the best in assisting virus transduction (FIG. 9). For the maleimide-chemistry approach, they tested the impact of different concentrations of Mal-FITC and found that cells were most strongly labeled with 5 μM Mal-FITC (FIG. 10). Given that all three labeling approaches (FIG. 5) are orthogonal, the inventors hypothesized that cell labeling with a combination of these labeling approaches could maximize the surface labeling with FITC and therefore likely enable the optimal virus transduction. Interestingly, NHS-chemistry-based solo labeling approach exhibited the best effect on promoting virus transduction, followed by NHS+amph (lipid-based) labeling approach, while including malemide-chemistry-based labeling tends to compromise virus transduction (FIG. 11). The inventors went on and confirmed that chemical labeling of K562 cells has minimal impact of cell viability, and the inclusion of polybrene reduced cell proliferation (FIG. 12). Finally, to understand the mechanisms by which each chemical labeling assists with virus transduction. They treated wt or chemically labeled K562 cells with methyl-β-cyclodextrin (MbCD), a potent inhibitor of clathrin-dependent endocytosis. While VSV-G dependent transduction is largely suppressed by MbCD, chemically labelling assisted virus transduction was only mildly impacted, indicating that chemical labeling might promote virus transduction through pathways that are independent of clathrin (FIG. 13).

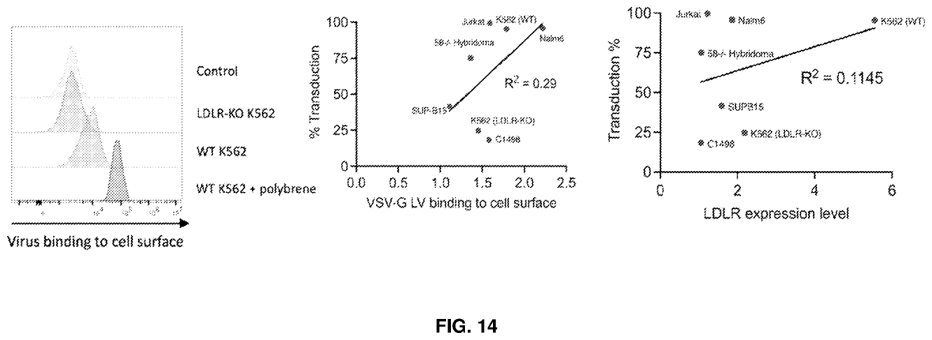

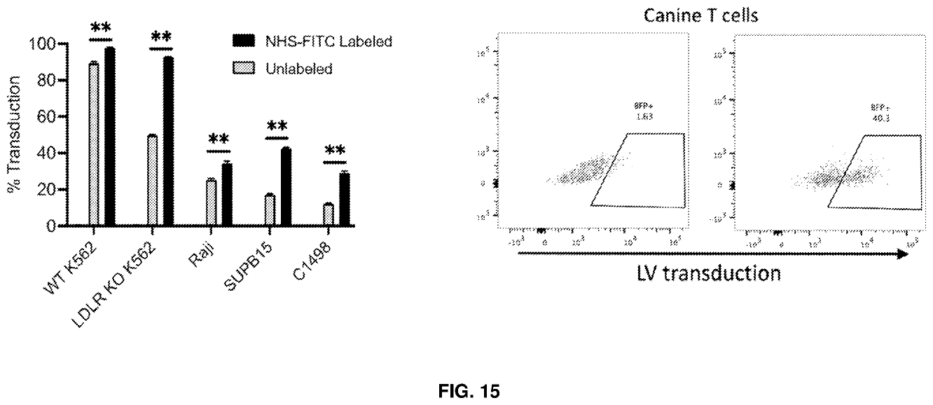

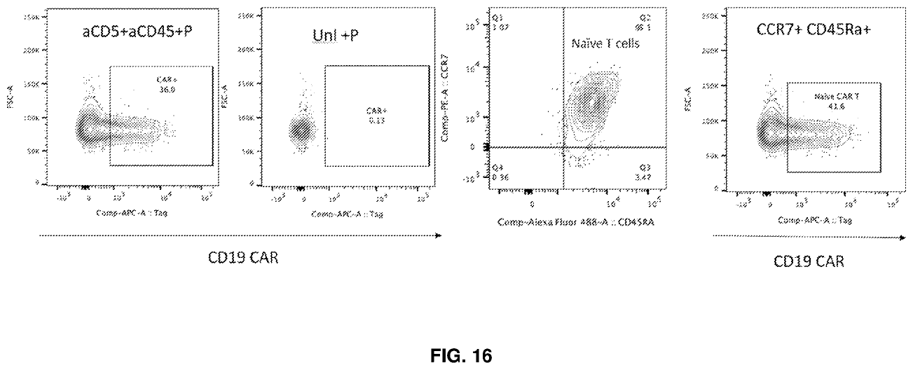

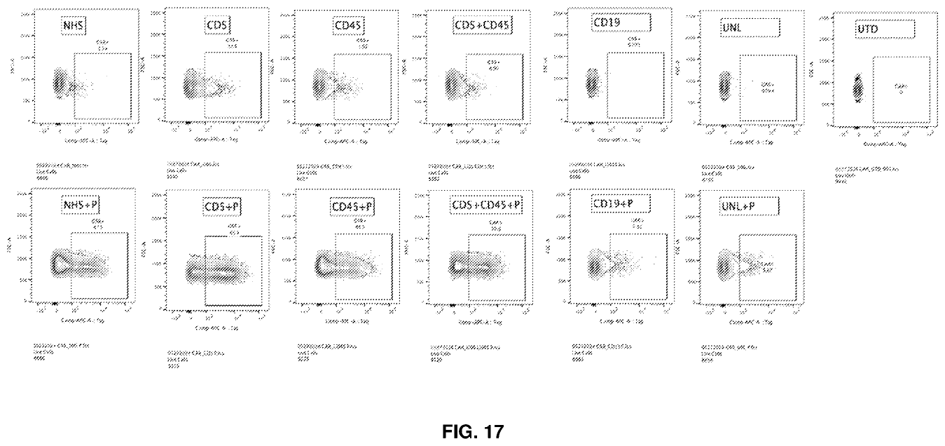

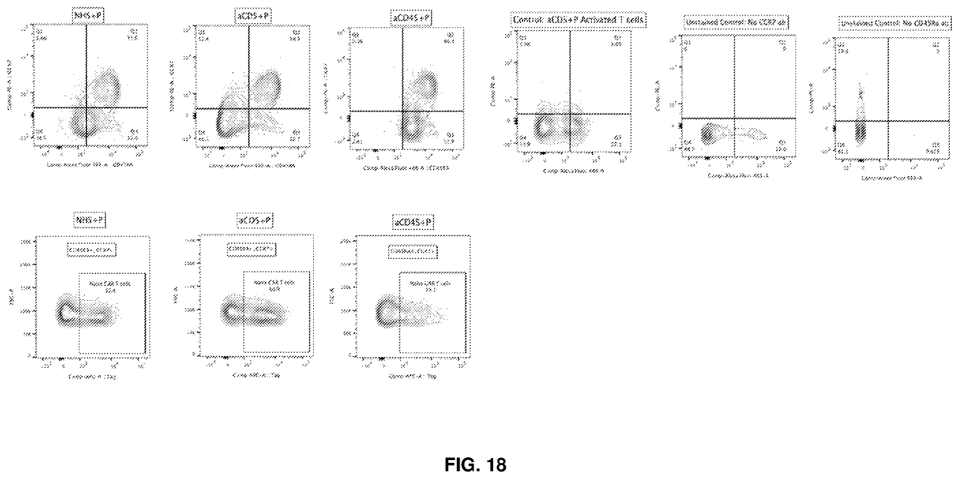

The inventors then tested the virus binding to WT and hLDLR-KO K562 cells and confirmed stronger virus binding to WT K562 cells, indicating this binding is strongly dependent on hLDLR (FIG. 14, left panel). They went on and tested the virus binding and hLDLR expression on a panel of mammalian cell lines, including SUP−B15, C1498, 58−/− hybridoma, Jurkat and NALM6. They found that virus transduction has a stronger correlation with virus binding than hLDLR expression (FIG. 14 mid, right panel), indicating other membrane proteins (e.g., LDLR family members) could assist virus binding and cell transduction. Chemically labeling improved virus transduction of all mammalian cell lines (FIG. 15, left panel) and canine T cells (FIG. 15, right panel). Finally, the inventors tested the transduction of naïve human T cells with a scFv-truncVSVG pseudotyped virus carrying a CD19 CAR followed by surface labeling using FITC-conjugated antibodies (anti-CD5, anti-CD45). CD19 CAR expression on naïve T cells was measured by flow cytometry 7 days post-transduction, and they found that >30% naïve T cells express the CD19 CAR. Notably, most transduced T cells maintained their naïve phenotype (CD45RA+, CCR7+) (FIG. 16). The inventors then compared NHS-chemistry-based labeling with FITC-conjugated antibody-based labeling (FITC-anti-CD19 included as control), and they found that FITC-anti-CD5 directed virus transduction performed the best in the presence of polybrene (FIG. 17). And all approaches could efficiently transduce (30-60%) naïve T cells (CD45RA+, CCR7+) (FIG. 18).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### IV. References

The following references, to the extent that they provide exemplary procedural or other details supplementary to those set forth herein, are specifically incorporated herein by reference.
Bischof et al., Flexibility in cell targeting by pseudotyping lentiviral vectors. Methods Mol Biol. 2010; 614:53-68.
Braasch and Corey, Chemistry & Biology 8, 1-7 (2001)
Chu et al., Efficient and Stable Gene Expression into Human Osteoclasts Using an HIV-1-Based Lentiviral Vector. DNA Cell Biol. 2008 June; 27(6): 315-320.
Coffin J M, Hughes S H, Varmus H E, Cold Spring Harbor (N.Y.): Cold Spring Harbour Laboratory Press; 1997; ISBN-10:0-87969-571-4.
Egholm et al., Nature 365:666 (1993).
Jacome et al., Lentiviral-mediated Genetic Correction of Hematopoietic and Mesenchymal Progenitor Cells from Fanconi Anemia Patients. Mol Ther. 2009 June; 17(6): 1083-1092.
Mátrai J, Chuah M K, VandenDriessche T. Recent advances in lentiviral vector development and applications. Mol Ther. 2010 March; 18(3):477-90.
Nielsen et al., Science 254:1497 (1991).
O'Connell R M, Balazs A B, Rao D S, Kivork C, Yang L, Baltimore D. Lentiviral vector delivery of human interleukin-7 (hIL-7) to human immune system (HIS) mice expands T lymphocyte populations. PLoS One. 2010 Aug. 6; 5(8):e12009.
Poczobutt et al., Benign mammary epithelial cells enhance the transformed phenotype of human breast cancer cells. BMC Cancer. 2010; 10: 373.
Sambrook and Russell “Molecular Cloning, A Laboratory Manual”, Cold Spring Harbor Laboratory, N.Y. (2001).
Vesicular stomatitis virus G glycoprotein pseudotyped retroviral vectors: concentration to very high titer and efficient gene transfer into mammalian and nonmammalian cells. Proc Natl Acad Sci USA. 1993; 90(17): 8033-8037.

1. A composition comprising a cell conjugated to fluorescein, wherein the cell has low or essentially no expression or undetectable expression of human low density lipoprotein receptor (hLDLR).

2. A composition comprising a cell expressing a VSV-G chimeric protein conjugated to conjugated to fluorescein.

3. The composition of claim 1, wherein the cell has low, undetectable or essentially no expression of human low density lipoprotein receptor (hLDLR), such as measured by flow cytometry.

4. The composition of claim 1, wherein the fluorescein is 5-carboxyfluorescein (5-FAM), FITC, or 6-carboxyfluorescein (6′FAM).

5. The composition of claim 1, wherein the fluorescein is NHS-5′FAM, Maleimide-5′FAM, or 1,2-Distearoyl-sn-glycero-3-phosphoethanolamine (DSPE)-Poly-ethylene-glycol(PEG)-Fluorescein.

6. The composition of claim 1, wherein the cell with low or essentially no expression of hLDLR is a human naïve T cell, resting human T cells, a human B cell, human epithelial cell, murine immune cell, or canine immune cell, monkey immune cell, or cancer cell line (e.g., SUP-B15 or Raji).

7. (canceled)

8. The composition of claim 1, wherein the 5-FAM is conjugated to N-hydroxysuccinimide at the 5′ carbon (NHS-5′-FAM) or an antibody targeting a cell surface protein.

9-10. (canceled)

11. The composition of claim 1, wherein the cell has been transduced with a pseudotyped lentivirus.

12-15. (canceled)

16. A nucleic acid encoding a VSV-G chimeric protein linked to an anti-fluorescein antibody or fragment thereof.

17. The nucleic acid of claim 16, wherein the nucleic acid has a sequence at least 90% identical to SEQ ID NO:2.

18. The nucleic acid of claim 16, wherein the anti-fluorescein antibody is an anti-fluorescein scFv.

19-20. (canceled)

21. The nucleic acid of claim 16, wherein the anti-fluorescein scFv is a 2A9M scFv derived from fluorescein targeting antibody FITC-E2.

22. The nucleic acid of claim 16, wherein the anti-fluorescein is fused to a VSV-G hinge, transmembrane and intracellular domain.

23. The nucleic acid of claim 16, wherein the fusion protein is under control of a constitutive promoter.

24. (canceled)

25. A vector comprising the nucleic acid of claim 16.

26. (canceled)

27. A host cell comprising the nucleic acid of claim 16.

28. A polypeptide encoded by the nucleic acid of claim 16.

29. A pseudotyped lentiviral vector particle comprising a VSV-G envelope and the polypeptide of claim 28.

30. A method for transducing a cell comprising contacting said cell with the pseudotyped lentiviral vector particles of claim 29 under conditions suitable for transduction, thereby transducing said cell.

31-41. (canceled)
